(19) United States
(12) Patent Application Publication
(10) Pub. No.: US 2008/0275236 A1
(43) Pub. Date: Nov. 6, 2008

(54) Method For Removing Salts From A Reaction Mixture Containing Alkoxycarbonyl Amino Triazine

(75) Inventors: Jorg Schneider, Wezembeek-Oppern (BE); Gunter Scherr, Ludwigshafen (DE); Rainer Erhardt, Mannheim (DE); Andreas Eichfelder, Maxdorf (DE); Martin Reif, Romerberg (DE); Stefan Hirsch, Neustadt an der Weinstrass (DE); Georg Sieder, Bad Durkheim (DE); Thomas Holtmann, Speyer (DE); Juergen Ciprian, Ludwigshafen (DE); Hermann Ascherl, Dirmstein (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 11/916,564

(22) PCT Filed: Jun. 1, 2006
(86) PCT No.: PCT/EP2006/062829

§ 371 Date: Dec. 5, 2007

(30) Foreign Application Priority Data
Jun. 6, 2005 (DE) 10 2005 025 900.6

(51) Int. Cl. C07D 251/00

(52) U.S. Cl. 544/218

(57) ABSTRACT

The invention relates to a process for removing salts from an alkanolic reaction mixture which is obtained in the preparation of alkoxycarbonylaminotriazines and comprises at least one alkoxycarbonylaminotriazine, at least one cyclic and/or acyclic carbonic ester, at least one C₁-C₁₃-alkanol which optionally comprises one or two oxygen atoms as ether bonds and is optionally substituted by C₁-C₄-alkyl and/or hydroxyl, and also at least one alkali metal alkoxide or alkaline earth metal alkoxide, with or without melamine and with or without catalyst, in which salts are removed from the reaction mixture by ion exchange over a cation exchanger and/or anion exchanger.

The invention relates to a process for removing salts from an alkanolic reaction mixture which is obtained in the preparation of alkoxycarbonylaminotriazines and comprises at least one alkoxycarbonylaminotriazine, at least one cyclic and/or acyclic carbonic ester, at least one C₁-C₁₃-alkanol which optionally comprises one or two oxygen atoms in the form of ether bonds and is optionally substituted by C₁-C₄-alkyl and/or hydroxyl, and also at least one alkali metal alkoxide or alkaline earth metal alkoxide, with or without melamine and with or without catalyst.

The preparation of alkoxycarbonylaminotriazines by reacting triazines, for example melamine, with carbonic esters in the presence of a base is known, for example, from EP-A 0 624 577. In this preparation, melamine is generally reacted with a carbonic ester in the presence of the parent alkanol of the carbonic ester and in the presence of an alkali metal alkoxide based on the parent alcohol of the carbonic ester as a base. For workup, a mineral acid is added to the reaction mixture for neutralization. Suitable acids mentioned are phosphoric acid, sulfuric acid and/or hydrochloric acid. The alkoxycarbonylaminotriazine is subsequently obtained by an extraction with an organic solvent and the evaporation of the solvent. Alternatively, after the addition of the acid, a solid is isolated by filtration and is then washed and dried.

WO-A 03/035628 discloses a process for preparing alkoxycarbonylaminotriazines, in which the reaction mixture is worked up by first neutralizing with a preferably aqueous acid. Suitable acids mentioned are nitric acid, sulfuric acid, phosphoric acid or mixtures thereof, but also formic acid. After the addition of the acid to the reaction mixture, an aqueous and an alkanolic phase are formed and are separated from one another. The alkanolic phase comprises the alkoxycarbonylaminotriazine. To increase the concentration of an alkoxycarbonylaminotriazine, the organic phase is concentrated after the removal of the aqueous phase.

A corresponding process for working up a reaction mixture comprising alkoxycarbonylaminotriazine is also disclosed in WO-A 2004/054990.

WO-A 2004/041922 discloses a preparation and workup process for carbamate-melamine-formaldehyde crosslinkers. In this process, the workup is likewise effected by addition of an acid, for example sulfuric acid, formic acid, oxalic acid, phosphoric acid, hydrochloric acid or mixtures thereof. The salt formed in neutralization is removed by filtration and washing with water.

It is an object of the present invention to provide a process which allows a substantially full removal of salts from an alkanolic reaction mixture comprising at least one alkoxycarbonylaminotriazine without a high level of apparatus complexity.

The object is achieved by a process for removing salts from an alkanolic reaction mixture which is obtained in the preparation of alkoxycarbonylaminotriazines and comprises at least one alkoxycarbonylaminotriazine, at least one cyclic and/or acyclic carbonic ester, at least one C₁-C₁₃-alkanol which optionally comprises one or two oxygen atoms as ether bonds and is optionally substituted by C₁-C₄-alkyl and/or hydroxyl, and also at least one alkali metal alkoxide or alkaline earth metal alkoxide, with or without melamine and with or without catalyst, in which salts are removed from the reaction mixture by ion exchange over a cation exchanger and/or anion exchanger.

Preferred alkoxycarbonylaminotriazines are those of the general formula (I)

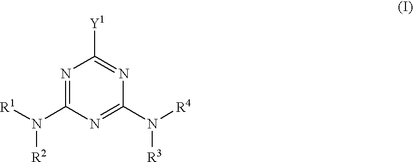

in which the symbols and indices are each defined as follows:

Y¹ is hydrogen, C₁-C₄-alkyl, phenyl optionally substituted by C₁-C₄-alkyl, C₁-C₄-alkoxy, or halogen, or a radical of the formula NR⁵R⁶ and

R¹, R², R³, R⁴, R⁵ and R⁶ are each independently hydrogen or a radical of the formula COOX or X, or selected from the group of (—CH₂—O)ₗ—H, (—CH₂—O)ₗ—R, (—CH₂—O)ₖ—CH₂—N(Z)-Q and (—CH₂—O)ₖ—CH₂—N(Z)-Q, where
k is from 0 to 10, preferably from 1 to 5, more preferably 1 or 2 and in particular 1, and l is from 1 to 10, preferably from 1 to 5, more preferably 1 or 2 and in particular 1,
R is selected from the group of alkyl, cycloalkyl and alkylaryl, where the R groups comprise preferably fewer than 13 carbon atoms and R is preferably a C₁-C₁₃-alkyl and more preferably methyl or butyl,
Q is a triazine radical of the general formula (II)

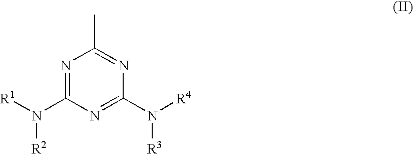

X is C₁-C₁₃-alkyl whose carbon skeleton may be interrupted by 1 or 2 nonadjacent oxygen atoms in ether function and/or substituted by hydroxyl, or is C₃-C₆-alkenyl and
Z is an R¹, R², R³, R⁴, R⁵ or R⁶ radical as defined above,
   and

at least one of the R¹ to R⁴ radicals or, when Y¹ is NR⁵R⁶, at least one of the R¹ to R⁶ radicals, is COOX.

C₁-C₄-Alkyl is, for example, methyl, ethyl, propyl, isopropyl, butyl, sec-butyl or tert-butyl.

Phenyl optionally substituted by C₁-C₄-alkyl, C₁-C₄-alkoxy or halogen is, for example, phenyl, 2-, 3- or 4-methylphenyl, 2-, 3- or 4-ethylphenyl, 2,4-dimethylphenyl, 2-, 3- or 4-methoxyphenyl, 2-, 3- or 4-ethoxyphenyl, 2,4-dimethoxyphenyl, 2-, 3- or 4-fluorophenyl or 2-, 3- or 4-chlorophenyl.

C₁-C₁₃-Alkyl whose carbon skeleton may be interrupted by 1 or 2 nonadjacent oxygen atoms in ether function and/or substituted by hydroxyl is, for example, pentyl, isopentyl, neopentyl, tert-pentyl, hexyl, 2-methylpentyl, heptyl, octyl, 2-ethylhexyl, isooctyl, nonyl, isononyl, decyl, isodecyl, undecyl, dodecyl, tridecyl, isotridecyl, 2-methoxyethyl, 2-ethoxyethyl, 2-propoxyethyl, 2-butoxyethyl, 2- or 3-methoxypropyl, 2- or 3-ethoxypropyl, 2- or 3-propoxypropyl, 2- or 4-methoxybutyl, 2- or 4-ethoxybutyl, 3,6-dioxaheptyl, 3,6-dioxaoctyl, 3,7-dioxaoctyl, 4,7-dioxaoctyl, 2- or 3-butoxypropyl, 2- or 4-butoxybutyl, 2-hydroxyethyl, 2- or 3-hydroxypropyl, 2- or 4-hydroxybutyl, 3-hydroxybut-2-yl. (The above names isooctyl, isononyl, isodecyl and isotridecyl are trivial names and stem from the alcohols obtained by the oxo process—cf. Ullmann's Encyclopedia of Industrial Chemistry, 5th Edition, Vol. A1, pages 290 to 293, and also Vol. A10, pages 284 and 285).

C₃-C₆-Alkenyl is, for example, allyl, methallyl, ethallyl, 2-, 3- or 4-penten-1-yl or 2-, 3-, 4- or 5-hexen-1-yl.

C₁-C₁₃-Alkanol which optionally comprises one or two nonadjacent oxygen atoms in the form of ether bonds and is optionally substituted by C₁-C₄-alkyl and/or hydroxyl is, for example, methanol, ethanol, propanol, isopropanol, butanol, isobutanol, sec-butanol, tert-butanol, pentanol, isopentanol, neopentanol, tert-pentanol, hexanol, 2-methylpentanol, heptanol, octanol, 2-ethylhexanol, isooctanol, nonanol, isononanol, decanol, isodecanol, undecanol, dodecanol, tridecanol, isotridecanol, 2-methoxyethanol, 2-ethoxyethanol, 2-propoxyethanol, 2-butoxyethanol, 2- or 3-methoxypropanol, 2- or 3-ethoxypropanol, 2- or 3-propoxypropanol, 2- or 4-methoxybutanol, 2- or 4-ethoxybutanol, 3,6-dioxaheptanol, 3,6-dioxaoctanol, 3,7-dioxaoctanol, 4,7-dioxaoctanol, 2- or 3-butoxypropanol, 2- or 4-butoxybutanol, ethane-1,2-diol, propane-1,2-diol, propane-1,3-diol, 3-oxa-5-hydroxypentanol, 3,6-dioxa-8-hydroxyoctanol, 3-oxa-5-hydroxy-2,5-dimethylpentanol or 3,6-dioxa-8-hydroxy-2,5,8-trimethyloctanol.

The C₁-C₁₃-alkanol which optionally comprises one or two nonadjacent oxygen atoms as an ether bond and is optionally substituted by C₁-C₄-alkyl- and/or hydroxyl is more preferably selected from methanol, ethanol, propanol, isopropanol, butanol, isobutanol, sec-butanol, tert-butanol, pentanol, isopentanol, neopentanol, tert-pentanol, hexanol, 2-methylpentanol and heptanol.

Very particular preference is given to butanol, isobutanol, sec-butanol and tert-butanol, and mixtures of methanol and butanol.

A cyclic carbonic ester is a carbonate of the general formula (III)

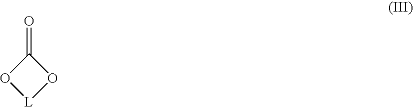

in which

L is ethylene, 1,2- or 1,3-propylene or 1,2-, 1,4-, 2,3- or 1,3-butylene.

Acyclic carbonic esters are, for example, diaryl carbonate, dialkyl carbonate, aryl alkyl carbonate and dialkenyl carbonate. The acyclic carbonic ester is preferably selected from carbonates of the general formula (IV)

Z¹O—CO—OZ² (IV)

in which

Z¹ and Z² are each independently alkyl, cycloalkyl and aryl. The Z¹ and Z² radicals preferably comprise fewer than 13 carbon atoms. More preferably, Z¹ and Z² are a C₁-C₈-alkyl and in particular methyl or butyl.

Preferred dialkyl carbonates are dimethyl carbonate, diethyl carbonate, dipropyl carbonate, dibutyl carbonate and methyl butyl carbonate.

Preferred aryl alkyl carbonates are methyl phenyl carbonate or butyl phenyl carbonate.

Suitable diaryl carbonates are, for example, diphenyl carbonate, di(para-tolyl) carbonate, di(α-naphthyl) carbonate or di(β-naphthyl) carbonate.

A preferred dialkenyl carbonate is diallyl carbonate.

Particularly preferred carbonic esters are dimethyl carbonate, diethyl carbonate, dibutyl carbonate, methyl butyl carbonate, diphenyl carbonate, propylene carbonate or mixtures thereof.

Suitable alkali metal or alkaline earth metal alkoxides are, for example, lithium, sodium, potassium, magnesium or calcium salts of the alkanols designated in detail above. The use of alkali metal methoxides, especially of sodium methoxide, is preferred. The alkali metal or alkaline earth metal alkoxide can be used either in the solid state or in dissolved or suspended form.

Preferred solvents/diluents are in this case especially the alcohols designated in detail above, alone or as a mixture with one another. However, other customary inert diluents known per se may also be used.

Catalysts which may be present in the reaction mixture are catalysts which are used to prepare the at least one alkoxycarbonylaminotriazine. Such catalysts are, for example, phase transfer catalysts, as described in Ullmann's Encyclopedia of Industrial Chemistry, 5th Edition, Vol. A19, pages 239 to 248. Further catalysts may be metal salts or complexes, preferably oxides, chalcogenates, carbonates or halides of the alkali metals, alkaline earth metals or transition metals. Mention should be made here in particular of lithium chloride, magnesium chloride or sodium carbonate.

In the reaction mixture, salts are generally present in dissociated form as ions. According to the invention, the salt ions are removed from the reaction mixture by ion exchange over a cation exchanger and/or anion exchanger.

Salts in the reaction mixture can form, for example, when acid is added to the alkanolic reaction mixture for neutralization or when the alkanolic reaction mixture is introduced into an acid. The acid may be in concentrated form or diluted with water. A uniform distribution of the acid in the reaction mixture is achieved by ensuring suitable mixing during the metered addition of the acid.

To neutralize the reaction mixture, all customary organic and inorganic acids used industrially may be used in any concentration, preferably as 30-85% by weight aqueous solutions. Preference is given to using mineral acids whose salts have a high water solubility, such as nitric acid, sulfuric acid or phosphoric acid. A further suitable acid is formic acid. According to the invention, particular preference is given to the use of nitric acid.

Anion exchangers suitable in accordance with the invention are, for example, strongly basic anion exchange resins. Preference is given to crosslinked polystyrene resins or styrene-divinylbenzene copolymers with tertiary or quaternary amines as a functional group and OH- ions as exchange ions. Exchange ions are understood to mean the ions which are bonded to the functional groups and are exchanged for the ions to be removed from the liquid. In commercially available anion exchangers, the functional groups are generally present as salts. In these anion exchangers, Cl⁻ ions, for example, are bonded to the functional group. In order to be able to use the anion exchangers, it is in this case generally first pretreated with NaOH in order to exchange the Cl⁻ ions or OH⁻ ions. Suitable commercially available anion exchangers are, for example, Lewatit® MP62, Lewatit® MP64 or Lewatit® MP 600 WS from Bayer AG, or else Amberjet® 4200 CL or Ambersep® 900 OH from Rohm & Haas Co. For the removal of nitrate ions, preference is given to Ambersep® 900 OH and Lewatit® MP 600 WS, particular preference to Ambersep® 900 OH.

Suitable cation exchangers are, for example, strongly acidic cation exchange resins based on a crosslinked polystyrene matrix or a styrene-divinylbenzene copolymer matrix and sulfonic acid as a functional group with H⁺ ions as exchange ions. In general, the cation exchangers, just like the anion exchangers, are in their salt form when available commercially. In order to be able to use the cation exchanger, it is then generally pretreated with an acid, for example sulfuric acid, in order to exchange the cations of the salt for H⁺ ions. Commercially available, suitable cation exchangers are, for example, Lewatit® S2528 or Lewatit MonoPlus® S100 from Bayer AG, Amberlyst® 40 WET and Amberjet® 1500 H from Rohm & Haas Co., and also Dowex® N306 from Dow Chemical Co. For the removal of sodium ions, for example, preference is given to using Amberlyst® 40 WET and Amberjet® 1500 H.

In one embodiment, the cation exchanger and/or the anion exchanger are present in the form of fixed bed ion exchangers. Instead of the fixed bed ion exchanger, in a further embodiment, the cation exchanger and/or the anion exchanger may also be present in the form of granules. In this case, the cation exchanger and/or the anion exchanger may be present in a vessel, for example a stirred tank, a column or in another apparatus known to those skilled in the art. The cation exchanger and/or anion exchanger are preferably present in a column.

In a further embodiment, it is also possible to feed the cation exchanger and/or anion exchanger as granules to the reactor in which the alkoxycarbonylaminotriazine is prepared. In this case, the reactor is preferably a stirred tank.

In a preferred embodiment, the alkali metal and/or alkaline earth metal ions are removed from the alkanolic reaction mixture with a cation exchanger.

It is also possible to remove anions of the salts with an anion exchanger. For example, as a result of the neutralization of the reaction mixture with an acid, the reaction mixture may comprise nitrate, sulfate or phosphate ions or else the anions of organic acids such as formic acid, which may be removed by the ion exchange with the anion exchanger.

Regeneration of the laden anion exchanger is effected preferably with dilute, mineral alkalis. Particularly suitable for the regeneration of the anion exchanger is 5-25% sodium hydroxide solution.

A regeneration of the cation exchanger is effected preferably with dilute mineral acids. A suitable mineral acid is, for example, 5-30% hydrochloric acid.

To pass through several cycles, both the anion exchange resin and the cation exchange resin are generally pretreated with a solubilizer between organic and polar phase. To this end, the ion exchanger is rinsed with a substance which has a polarity which is between the polarity of the organic phase and of the polar phase and is preferably miscible with both phases. For example, methanol is suitable as a solubilizer when butanol is the organic phase and water is the polar phase. In addition to a regeneration of the ion exchange resin, it is also conceivable to discard the laden resin without regeneration.

The cation exchanger and the anion exchanger may be used either together in the form of a mixture, individually or in steps or stages connected in series. Suitable combinations of suitable commercially available anion exchangers and cation exchangers are, for the removal of nitrate salts, Ambersep® 900 OH or Amberjet® 4200 as the anion exchanger and Lewatit® S2528 as the cation exchanger. Preference is given to the combination of Ambersep® 900 OH and Lewatit® S2528.

The alkanolic reaction mixture can be contacted with the cation exchanger and/or anion exchanger, for example, by adding the cation exchanger and/or anion exchanger to the reaction mixture, for example into the reactor or into a stirred vessel, or by flowing the reaction mixture through a continuous ion exchanger, in which case the ion exchanger is present, for example, as a packing in a fixed bed.

The addition of the ion exchange resin into the reaction vessel is possible especially when the at least one alkoxycarbonylaminotriazine is prepared batchwise. In this case, preference is given to effecting both the preparation of the at least one alkoxycarbonylaminotriazine and, if appropriate, a neutralization of the reaction mixture and the removal of the salts by ion exchange in the same vessel.

In the ion exchange, OH groups or hydrogen ions are generally exchanged for the anions or cations present in the mixture. The loss of the OH groups or hydrogen ions changes the pH in the mixture. In order to obtain a desired pH in the mixture comprising the at least one alkoxycarbonylaminotriazine, an acid can be added to it to lower the pH or a base can be added to it to increase the pH. The acid or the base can be added before, during or after the ion exchange.

Suitable acids for adjusting the pH are all mineral or organic acids. In order not to counteract the effect of the ion exchange, the acids used are generally not those whose ions correspond to the ions removed with the ion exchanger. For example, in the case of removal of nitrate ions from the mixture, nitric acid should not be used to adjust the pH. The pH is preferably adjusted by using organic acids. Particular preference is given to formic acid.

The bases conceivable for adjustment of the pH are any bases. Here too, it should be noted that bases whose ions correspond to the ions removed by the ion exchange should not be used. A preferred base for adjusting the pH is aqueous ammonia.

In one embodiment, a portion of the salts is removed from the alkanolic reaction mixture before the ion exchange by washing, extraction or filtration or combinations thereof.

The washing is effected preferably by adding water at a temperature in the range from 10 to 70° C., preferably from 15 to 50° C., and at a pH of from 0 to 8, preferably from 2 to 5.

The extraction is preferably carried out with a polar extractant which is not entirely miscible with the organic phase to obtain an alkanolic phase comprising alkoxycarbonylaminotriazine and a polar phase comprising extractant with salts dissolved therein. In this context, not entirely miscible means that two phases with different composition form, and not entirely miscible is also understood to mean that the extractant and the organic phase do not mix at all. A preferred extractant is water, and particular preference is given to fully demineralized water.

In addition to the polar extractant, separating assistants may be added in the extraction. Suitable separating assistants are, for example, organic solvents or those as described, for example, in Ullmann's Encyclopedia of Industrial Chemistry, Sixth Edition, 2000 Electronic Release, Section: Emulsion, Chapter 6, Breaking of Emulsions.

For the extraction, the apparatus known to those skilled in the art, for example mixer/settler units, columns with or without energy input or extractors which are based on the principle of centrifugal field separation, may be used. A mixer/settler unit comprises generally a mixer unit such as a stirred vessel, a mixing pump, a nozzle or a static/dynamic mixer. The mixer/settler unit further comprises a separator which is generally designed as a horizontal vessel with or without internals.

Suitable columns which can be used for the extraction are, for example, columns with structured packing or random packing, or sieve tray columns. Suitable sieve tray columns are, for example, also crossflow sieve tray columns. The random packings used may be all random packings known to those skilled in the art. Such random packings are described, for example, in Klaus Sattler, Thermische Trennverfahren [Thermal separating processes], 2nd Edition, VCH Verlagsgesellschaft mbH, Weinheim, 1995, pages 226 to 229.

Suitable structured packings are ordered or unordered packings. Such structured packings are, for example, lamella packings, fabrics, drawn-loop knits or formed-loop knits.

The random packings, structured packings or sieve trays may be manufactured from metal or plastic. Owing to the good wetting properties of metals, preference is given to using metal as the material for the random packings, structured packings or sieve trays when the wash phase is selected as the continuous phase. Particularly suitable metals are stainless steels.

In addition to the operation of the columns with random packings, structured packings or trays with and without pulsation, it is also conceivable to use them without internals, for example as a spray column. Examples of suitable commercial extraction columns with mechanical stirrer systems are rotary disk extractors, Old-Rushton columns, Kühni extractors, stirred cell extractors, Graesser extractors. However, it is also possible to use centrifugal extractors, for example Podbielniak extractors or Lurgi-Westfalia extractors.

In the extraction, the wash phase comprising the polar extractant which is not entirely miscible with the organic phase may form either the continuous or the disperse phase of the extraction. In a preferred form of the extraction, the wash phase forms the continuous phase.

In addition to the use of a single extraction apparatus, it is also possible to carry out the extraction in a plurality of apparatuses. In this case, a combination of different apparatus types may also be used. A preferred combination is formed by a mixer/settler unit and a column with random packing. In a particularly preferred embodiment, the extraction is carried out in a column with structured packing. In the extraction, the phase ratio of polar to organic phase is within a range from 0.1 to 2. Preference is given to a phase ratio in the range from 0.15 to 1.5, more preferably from 0.2 to 1 and in particular from 0.3 to 0.5.

When a column with structured packing is used for extraction, in a preferred embodiment, the polar phase is drawn off as the extract via the bottom of the column; the raffinate, the organic phase, preferably runs off as a free overflow. The extraneous phase content, i.e. the alkanol, in the extract is preferably removed by a drawn-loop plastics knit in the bottom of the column.

When water is used as the polar extractant, the extraneous phase content, i.e. the content of undissolved water, in the raffinate after the phase separation is generally about 1 percent.

The raffinate comprises the desired product. Should the content of polar and ionic components in the raffinate be greater than the required product specification allows, it is possible in a preferred embodiment to recycle the raffinate into the feed. In this case, the raffinate may, for example, either be fed directly into the feed to the extraction or recycled into a buffer vessel from which the extraction is fed.

The polar and ionic components are, for example, alkali metal or alkaline earth metal salts, alkali metal or alkaline earth metal alkoxides, acid, cyclic or acyclic mono- and/or diesters of carbonic acid, alkan(edi)ols and also polar melamine derivatives. Alkanediols are, for example glycol and propanediol; polar melamine derivatives are, for example, melamine, mono- and dialkoxycarbonylaminotriazines.

The possibility of removing mono- and dialkoxycarbonylaminotriazines from the raffinate by the extraction makes it possible to adjust, in a controlled manner, the ratios of different alkoxycarbonylaminotriazines in the raffinate.

The extraction in the column with structured packing is carried out generally as a countercurrent extraction. In a particularly preferred embodiment, this is done by feeding the polar extractant above the packing and the alkanolic reaction mixture below the packing. Within the column, the polar extractant thus flows through the packing in the direction of the column bottom and the alkanolic reaction mixture through the packing in the direction of the column top. The alkanolic reaction mixture and the polar extractant mix in the packing, and the salts present in the alkanolic reaction mixture are passed to the polar extractant and thus removed from the alkanolic reaction mixture.

The temperature at which the extraction is carried out is preferably in the range from 10 to 90° C., more preferably in the range from 15 to 50° C.

A preferred pressure at which the extraction is carried out is ambient pressure. However, it is also possible to carry out the extraction at a pressure below ambient pressure or else at an elevated pressure. When the extraction is carried out at elevated pressure, the pressure is preferably in the range from 1 to 10 bar.

In one embodiment, the process may, as an additional step, comprise the concentration of the organic phase comprising at least one alkoxycarbonylaminotriazine.

The concentration can be effected by thermal or mechanical processes. Suitable thermal processes for the concentration are, for example, evaporation, distillation, rectification, drying, preferably spray drying or crystallization. Suitable mechanical processes are in particular membrane separation processes, for example pervaporation or permeation, and also filtration when the at least one alkoxycarbonylaminotriazine is present as a suspension. The processes for the concentration may each be employed individually or in combination. It is also possible to use any further process for concentration known to those skilled in the art. Preferred processes for concentration are distillation and spray drying.

In the case of concentration by distillation, it can be performed either before the ion exchange or after the ion exchange. In a preferred embodiment, the concentration by distillation is effected before the ion exchange. In this context, it has been found to be advantageous that the distillation reduces the volume stream comprising the at least one alkoxycarbonylaminotriazine and thus smaller apparatus can be used to carry out the ion exchange. This lowers both the capital and the operating costs for the ion exchange.

The concentration of the organic phase comprising at least one alkoxycarbonylaminotriazine by distillation can be effected continuously or batchwise.

For the continuous distillation, it is possible to use conventional continuous evaporators known to those skilled in the art. Suitable evaporators for continuous distillation are, for example, circulation evaporators such as Robert self-circulation evaporators, rapid-circulation evaporators with inclined evaporator tubes, forced-circulation evaporators with external evaporator bundles, circulation evaporators with boiling space divided into chambers, or forced-circulation evaporators with horizontal heater. Further suitable continuous evaporators are, for example, falling-film evaporators, thin-film evaporators or Kestner evaporators.

In addition, the organic phase can be concentrated by distillation in a column. The heating to evaporation temperatures can be effected at the bottom of the column or in a heat exchanger disposed outside the column. Suitable columns are, for example, columns with structured packing or random packing, or tray columns. Suitable structured packings, random packings or trays are all structured packings, random packings or trays known to those skilled in the art.

Batchwise concentration by distillation can be effected, for example, in a stirred vessel. The distillation can also be carried out in the vessel in which the reaction to give alkoxycarbonylaminotriazine is carried out. Preference is given to effecting the concentration by distillation in an additional stirred vessel.

Both in the continuous and in the batchwise process, the product stream comprising the at least one alkoxycarbonylaminotriazine is obtained as the liquid phase and a vapor stream comprising at least one alkanol, carbonate and water. When, in addition to ion exchange, an extraction is carried out before the distillation, and a polar extractant different from water is used, the polar extractant is present in the vapor either instead of the water or in addition.

The concentration of the organic phase comprising at least one alkoxycarbonylaminotriazine by distillation leads, in a particularly preferred embodiment, to a product stream which comprises 45-60% by weight of alkoxycarbonylaminotriazine.

However, depending on the desired product stream, it is also possible for the distillation to afford a product stream which comprises a smaller or else a larger proportion of alkoxycarbonylaminotriazine.

In a further process variant, in the concentration of the organic phase comprising at least one alkoxycarbonylaminotriazine, the organic phase and the polar phase are removed by distillation. To this end, the organic phase comprising at least one alkoxycarbonylaminotriazine is fed to a distillation column. In this case, the distillation column preferably comprises a rectifying section and a stripping section. The organic phase comprising at least one alkoxycarbonylaminotriazine is fed preferably via a side feed in the rectifying section.

At the bottom of the distillation column, the concentrated organic phase comprising at least one alkoxycarbonylaminotriazine is obtained.

In a preferred embodiment, the thus obtained bottom product is utilized in a heat exchanger to heat the desalted organic phase which is fed as feed to the distillation column.

Via the top of the distillation column, alkanols, if appropriate low boilers and water and/or polar extractant are drawn off. In a particularly preferred embodiment, this stream is subsequently fed to a phase separator in which the polar phase is separated from the organic phase. The organic phase is preferably fed as reflux back to the distillation column at the top thereof.

In a particularly preferred embodiment, the distillation column comprises a side draw arranged preferably in the stripping section, via which a preferably vaporous and substantially anhydrous stream comprising carbonate and alkanol is drawn off. A particularly preferred position of the side draw is directly above the column bottom or directly below the separating internals in the column.

Advantages of this operating mode lie in the recovery of a majority of the carbonates via side draw for reuse in the reaction, the reduction in the carbonate content in the product stream, and in the recovery of anhydrous high-boiling alkanols, for example n-butanol, via the side draw.

In a preferred embodiment, the distillation column comprises from 8 to 22 theoretical plates.

The reflux ratio of the organic phase at the top of the column is preferably in the range between 0.2 and 3 kg/kg.

The distillation column is preferably operated with a pressure in the range between 20 and 2000 mbar at the top of the column. The preferred range in which the column is operated is between 50 and 950 mbar.

In a further process variant, it is possible, in addition to the concentration of the phase comprising at least one alkoxycarbonylaminotriazine by distillation or instead of the concentration by distillation, to provide a spray drying as a further process step. The spray drying generates pulverulent alkoxycarbonylaminotriazine.

Preference is given to effecting the spray drying in a spray dryer as is known to those skilled in the art. For example, commercial spray dryers with atomizer disk, one-substance nozzle or two-substance nozzle may be used for the spray drying. Depending on the design, operation can be effected in cocurrent or in countercurrent. Preference is given to using a two-substance nozzle in which the liquid phase comprising at least one alkoxycarbonylaminotriazine is atomized at ambient pressure with the aid of a nitrogen stream. The nitrogen stream has a pressure in the range from 1 to 10 bar, preferably in the range from 2 to 5 bar and more preferably in the range from 2.5 to 5 bar. The nitrogen gas is used preferably as the cycle gas.

The spray drying is carried out preferably at a temperature in the range from 50 to 250° C., preferably at a temperature of from 55 to 150° C. and more preferably at a temperature in the range from 6 to 100° C., and at ambient pressure or an elevated pressure or reduced pressure of up to +/−0.01 MPa based on ambient pressure.

The pulverulent product generated in the spray drying may be removed, for example, in a fabric filter of customary design, such as candle filters, bag filters, hose filters or other filters known to those skilled in the art, or in a cyclone. Suitable filter material for a fabric filter is, for example, polytetrafluoroethylene, silicone or polyester. Preference is given to polyester.

The nitrogen used as the cycle gas is purified preferably in a scrubber. It is possible to use any scrubber known to those skilled in the art.

### EXAMPLES

### Example 1

100 ml of a 50% by weight butanolic reaction mixture comprising alkoxycarbonylaminotriazine, which had been neutralized with 30% nitric acid and subsequently washed, was admixed with 5.078 g of Amberjet 4200 anion exchange resin and agitated at a temperature of 30° C. for 18 h. This reduced the nitrate ion content in the reaction mixture from 0.26 g/100 g of reaction mixture to 0.026 g/100 g of reaction mixture.

### Example 2

The same reaction mixture as in Example 1 was agitated with 5.271 g of Ambersep 900 OH anion exchange resin at a temperature of 30° C. for 18 h. This reduced the nitrate ion content from 0.26 g/100 g of reaction mixture to 0.003 g/100 g of reaction mixture.

### Example 3

Here, the anion exchange resin from Example 1 was exchanged for 5.119 g of MP 600 WS anion exchange resin. This reduced the nitrate ion content from 0.26 g/100 g of reaction mixture to 0.031 g/100 g of reaction mixture.

### Example 4

The anion exchange resin used in Example 1 was replaced by 5.570 g of MP 62 anion exchange resin. This reduced the nitrate ion content from 0.26 g/100 g of reaction mixture to 0.16 g/100 g of reaction mixture.

### Example 5

The anion exchange resin from Example 1 was replaced by 5.101 g of MP 64 anion exchange resin. This reduced the nitrate ion content from 0.26 g/100 g of reaction mixture to 0.1 g/100 g of reaction mixture.

### Example 6

The anion exchange resin from Example 1 was replaced by 5.140 g of MP 64 anion exchange resin and 5.016 g of Lewatit S2528 cation exchanger. This reduced the nitrate ion content from 0.26 g/100 g of reaction mixture to 0.037 g/100 g of reaction mixture, and the sodium ion content from 830 mg/kg of reaction mixture to 195 mg/kg of reaction mixture.

### Example 7

The ion exchanger from Example 6 was replaced by 5.029 g of MP 62 anion exchange resin and 5.186 g of Lewatit S2528 cation exchanger. This reduced the nitrate ion content from 0.26 g/100 g of reaction mixture to 0.063 g/100 g of reaction mixture, and the sodium ion content from 830 mg/kg of reaction mixture to 220 mg/kg of reaction mixture.

### Example 8

The ion exchanger from Example 6 was replaced by 5.004 g of MP 600 WS anion exchange resin and 5.076 g of Lewatit S2528 cation exchanger. This reduced the nitrate ion content from 0.26 g/100 g of reaction mixture to 0.016 g/100 g of reaction mixture, and the sodium ion content from 830 mg/kg of reaction mixture to 150 mg/kg of reaction mixture.

### Example 9

The ion exchanger from Example 6 was replaced by 5.019 g of Ambersep 900 OH anion exchange resin and 5.089 g of Lewatit S2528 cation exchanger. This reduced the nitrate ion content from 0.26 g/100 g of reaction mixture to 0.001 g/100 g of reaction mixture, and the sodium ion content from 830 mg/kg of reaction mixture to 95 mg/kg of reaction mixture.

### Example 10

The ion exchanger from Example 6 was replaced by 5.002 g of Amberjet 4200 anion exchange resin and 5.005 g of Lewatit S2528 cation exchanger. This reduced the nitrate ion content from 0.26 g/100 g of reaction mixture to <0.001 g/100 g of reaction mixture, and the sodium ion content from 830 mg/kg of reaction mixture to 175 mg/kg of reaction mixture.

### Example 11

100 ml of a non-neutralized reaction mixture comprising alkoxycarbonylaminotriazine was agitated at 60° C. for 4 days with 50.552 g of Lewatit S2528 cation exchanger. A reduction in the sodium ion content from 30 000 ppm to 140 ppm was observed. The cream-like reaction mixture became liquid on addition of ion exchanger and no color change in the solution occurred.

### Example 12

The cation exchanger from Example 11 was replaced by 49.363 g of MonoPlus S100 cation exchanger. A reduction in the sodium ion content from 30 000 ppm to 280 ppm was observed. The cream-like reaction mixture became liquid on addition of ion exchanger and a color change of the solution to a distinctly brown hue occurred.

### Example 13

The cation exchanger from Example 11 was replaced by 51.136 g of Amberlyst 40 WET cation exchanger. A reduction in the sodium ion content from 30 000 ppm to 20 ppm was observed. The cream-like reaction mixture became liquid on addition of ion exchanger and the solution became distinctly yellowier.

### Example 14

The cation exchanger from Example 11 was replaced by 50.692 g of Amberjet 1500 H cation exchanger. A reduction in the sodium ion content from 30 000 ppm to 10 ppm was observed. The cream-like reaction mixture became liquid on addition of ion exchanger and the solution became distinctly yellowier.

### Example 15

The cation exchanger from Example 11 was replaced by 50.096 g of Dowex N306 cation exchanger. A reduction in the sodium ion content from 30 000 ppm to 40 ppm was observed. The cream-like reaction mixture became liquid on addition of ion exchanger and the solution became white.

### Example 16

To assess the influence of the pretreatment of Ambersep 900 OH, 5.036 g of Ambersep 900 OH were washed with fully demineralized water and agitated at a temperature of 30° C. with 100 ml of a reaction mixture comprising alkoxycarbonylaminotriazine for 24 h. The analysis yielded a reduction in the nitrate ion content from 0.26 g/100 g of reaction mixture to 0.002 g/100 g of reaction mixture.

### Example 17

5.068 g of Ambersep 900 OH were washed with fully demineralized water and subsequently with methanol. The anion exchanger was then agitated at a temperature of 30° C. with 100 ml of a reaction mixture comprising alkoxycarbonylaminotriazine for 24 h. The analysis yielded a reduction in the nitrate ion content in the reaction mixture from 0.26 g/100 g of reaction mixture to 0.013 g/100 g of reaction mixture.

### Example 18

5.077 g of Ambersep 900 OH were washed with fully demineralized water and subsequently with butanol. The anion exchanger was then agitated at a temperature of 30° C. with 100 ml of a reaction mixture comprising alkoxycarbonylaminotriazine for 24 h. The analysis yielded a reduction in the nitrate ion content from 0.26 g/100 g of reaction mixture to 0.005 g/100 g of reaction mixture.

### Example 19

5.123 g of Ambersep 900 OH were washed with fully demineralized water and subsequently first with methanol and then with butanol. The anion exchanger was then agitated at a temperature of 30° C. with 100 ml of neutralized reaction mixture comprising alkoxycarbonylaminotriazine for 24 h. The analysis yielded a reduction in the nitrate ion content from 0.26 g/100 g of reaction mixture to 0.018 g/100 g of reaction mixture.

### Example 20

To assess the influence of the residual water content of the end product on the liquid phase adsorption with Ambersep 900 OH, 3.183 g of zeolite 3A were agitated with 100 ml of a neutralized reaction mixture comprising alkoxycarbonylaminotriazine for 4 h. Afterward, the reaction mixture was admixed with 5.133 g of Ambersep 900 OH and agitated at a temperature of 30° C. for a further 24 h. The analysis yielded a reduction in the nitrate ion content from 0.26 g/100 g of reaction mixture to 0.001 g/100 g of reaction mixture.

### Example 21

10.998 g of zeolite 3A were agitated with 100 ml of a neutralized reaction mixture comprising alkoxycarbonylaminotriazine for 4 h. Afterward, the reaction mixture was admixed with 5.091 g of Ambersep 900 OH and agitated at a temperature of 30° C. for a further 24 h. The analysis yielded a reduction in the nitrate ion content from 0.26 g/100 g of reaction mixture to 0.001 g/100 g of reaction mixture.

### Example 22

10.007 g of zeolite 3A were agitated with 100 ml of neutralized reaction mixture comprising alkoxycarbonylaminotriazine for 4 h. Afterward, the reaction mixture was admixed with 1.156 g of Ambersep 900 OH and agitated at a temperature of 30° C. for a further 24 h. The analysis yielded a reduction in the nitrate ion content from 0.26 g/100 g of reaction mixture to 0.150 g/100 g of reaction mixture.

### Example 23

Two fixed bed columns connected in series were operated with 160 ml/h of neutralized reaction mixture comprising alkoxycarbonylaminotriazine at 30° C. for 7.5 h. The reaction mixture was circulated by means of a metering pump. The loading of nitrate ions in the reaction mixture was 0.25 g/100 g of reaction mixture. Fixed bed column 1 had a diameter of 10 mm, a bed height of 470 mm and was charged with Ambersep 900 OH anion exchange resin. Fixed bed column 2 had a diameter of 10 mm, a bed height of 490 mm and was charged with a zeolite 3A molecular sieve. The residence time in the columns was 15 min and the superficial velocity 2 m/h. Before the operation of the fixed bed columns, the anion exchanger fixed bed was flushed with 20 bed volumes of fully demineralized water and subsequently with 16 bed volumes of n-butanol. The breakthrough of nitrate ions occurred after approx. 40 min. The loading of the ion exchange resin was 0.36 eq/l.

### Example 24

A fixed bed column was operated with a volume stream of 160 ml/h of neutralized reaction mixture comprising alkoxycarbonylaminotriazine for 24 h. The reaction mixture was circulated by means of a metering pump. The nitrate ion content in the reaction mixture was 690 ppm. The fixed bed column had a diameter of 20 mm, a bed height of 955 mm and was charged with Ambersep 900 OH. The residence time was 2 h, the superficial velocity 0.5 m/h. Before the operation of the fixed bed column with the reaction mixture, the fixed bed was flushed with 20 bed volumes of fully demineralized water and subsequently displaced with 4.25 bed volumes of n-butanol for 8 h. The nitrate ion content in the reaction mixture leaving the fixed bed was always below 10 ppm. After the 24 h had elapsed, the reaction mixture was displaced with 1.6 bed volumes of n-butanol over 3 h. Subsequently, the fixed bed was washed with 6.8 bed volumes of fully demineralized water for 2 h and subsequently regenerated with 5.1 bed volumes of 5% sodium hydroxide solution. Next, the fixed bed column was washed to free it of alkali with 6.8 bed volumes of fully demineralized water for 2 h and then rinsed to free it of water with 6 bed volumes of n-butanol for 7 h. The fixed bed column was then operated with a volume stream of 160 ml/h of neutralized reaction mixture comprising alkoxycarbonylaminotriazine and having a nitrate ion content of 850 ppm for 50 h. Before the breakthrough, the nitrate ion contents were below 10 ppm. Up to the start of breakthrough, a capacity of 0.18 eq/l was achieved on the anion exchange resin.

1. A process for removing salts from an alkanolic reaction mixture which is obtained in the preparation of alkoxycarbonylaminotriazines and comprises at least one alkoxycarbonylaminotriazine, at least one cyclic and/or acyclic carbonic ester, at least one C₁-C₁₃-alkanol which optionally comprises one or two oxygen atoms in the form of ether bonds and is optionally substituted by C₁-C₄-alkyl and/or hydroxyl, and also at least one alkali metal alkoxide or alkaline earth metal alkoxide, with or without melamine and with or without catalyst, in which salts are removed from the reaction mixture by ion exchange over a cation exchanger and/or anion exchanger.

2. The process according to claim 1, wherein the cation exchanger and/or the anion exchanger are present in the form of fixed bed ion exchangers.

3. The process according to claim 1, wherein the cation exchanger and/or the anion exchanger are present in the form of granule.

4. The process according to claim 1, wherein the anion exchanger is a strongly basic anion exchange resin based on a crosslinked polystyrene matrix or styrene-divinylbenzene copolymer matrix with tertiary or quaternary amines as a functional group and OH- ions as exchange ions.

5. The process according to claim 1, wherein the cation exchanger is a strongly acidic cation exchange resin based on a crosslinked polystyrene matrix or a styrene-divinylbenzene copolymer matrix with sulfonic acid as a functional group and H⁺ ions as exchange ions.

6. The process according to claim 1, wherein the cation exchange resin and/or anion exchange resin is pretreated with a solubilizer between organic and polar phase.

7. The process according to claim 1, wherein the cation exchanger and the anion exchanger are in the form of a mixture.

8. The process according to claim 1, wherein the cation exchanger and the anion exchanger are used in steps connected in series.

9. The process according to claim 1, wherein the pH of the reaction mixture is adjusted by adding acid or base before, during or after the ion exchange.

10. The process according to claim 1, wherein, before the ion exchange, a portion of the salts is removed from the reaction mixture by washing, extraction or filtration or combinations thereof.

11. The process according to claim 1, which further comprises, the concentration of the organic phase comprising alkoxycarbonylaminotriazine.

12. The process according to claim 11, wherein the concentration of the organic phase comprising alkoxycarbonylaminotriazine is performed before the ion exchange.

13. The process according to claim 10, wherein the washing or the extraction of the neutralized reaction mixture is performed with water.

14. The process according to claim 1, wherein the alkali metal alkoxide or alkaline earth metal alkoxide is sodium methoxide.

15. The process according to claim 1, wherein the C₁-C₁₃-alkanol is butanol.

16. The process according to claim 1, wherein the at least one C₁-C₁₃-alkanol which optionally comprises one or two oxygen atoms as ether bonds and is optionally substituted by C₁-C₄-alkyl and/or hydroxyl is a mixture of methanol and butanol.

17. The process according to claim 1, wherein the alkanolic reaction mixture is neutralized before the ion exchange by addition of an acid.

18. The process according to claim 17, wherein the acid added for neutralization is nitric acid.

19. The process according to claim 1, wherein the process further comprises, the preparation of a powder comprising substantially alkoxycarbonylaminotriazine by spray drying.

20. The process according to claim 19, wherein the spray drying is carried out at a temperature in the range from 50 to 250° C.
